(19) United States
(12) Patent Application Publication
(10) Pub. No.: US 2003/0203016 A1
(43) Pub. Date: Oct. 30, 2003

(54) Freeze-dried agent containing paramylon, its production and use

(76) Inventors: Wolfgang Suwelack, Billerbeck (DE); Petra Tewes-Schwarzer, Senden (DE); Dietrich Baumer, Spenge (DE); Uwe Kahmann, Oerlinghausen (DE)

Correspondence Address: Connolly Bove Lodge & Hutz LLP, P.O. Box 2207, Wilmington, DE, 19899-2207, US

(21) Appl. No.: 10/437,686

(22) Filed: May 12, 2003

Related U.S. Application Data
(63) Continuation of application No. 09/890,980, filed on Aug. 8, 2001, now abandoned.
(63) Continuation of application No. 09/880,637, filed on Jun. 13, 2001, now abandoned.

(30) Foreign Application Priority Data
Feb. 10, 1999 (EP) EP 99 102 499.3

(51) Int. Cl. C12P 21/02; C07K 14/44; A61K 9/70

(52) U.S. Cl. 424/449; 435/69.1; 530/395

(57) ABSTRACT

The invention relates to an agent characterized in trhat it comprises 0.1 to 100 weight percent of freeze-dried paramylon, ist production and use.

The present invention relates to a freeze-dried agent comprising paramylon, for topical, parenteral or peroral administration, to a method for the production of the above-mentioned agent as well as to the use of this agent as a biomatrix, especially as a plaster, as a nutritional supplement, and for the administration of cosmetics or pharmaceuticals.

State of the Art

U.S. Pat. No. 5,158,772, for example, discloses topical, cosmetic and pharmaceutical compositions for use on the skin, said compositions containing as the carrier a β-1,3-glucan polysaccharide polymer which constitutes the reserve carbohydrate from Cellulomonas flavigena or its genetic clones. On the basis of its IR- and NMR-spectroscopic data, this β-1,3-glucan belongs to the subgroup of the curdlan polysaccharides but, unlike these, whose degree of polymerization ranges from 200 to 400, it exhibits a divergent value of about 550.

EP-B 417,254 (=U.S. Pat. No. 5,385,832) discloses the production and further processing of a β-1,3-glucan obtained from Euglena cells whereby, after cultivation, the cells are separated from the culture supernatant, the cells are destroyed and extracted using an organic solvent, after which the cell mass is separated and the resultant β-1,3-glucan is washed. This β-1,3-glucan should be useable in medicine as well as in cosmetics or as a food product. The production method according to the invention differs from this by avoiding the toxic organic extraction agents, such as methanol and chloroform, and yields the β-1,3-glucan paramylon already after a few days at a purity of usually more than 99% to 99.5% by weight, which is well above the purity of the glucan obtained through extraction with solvents, namely, only above 95% to 97%. A duplication of the single embodiment example, as discussed below, also showed a cell growth that is completely inadequate for a technically realistic cultivation method in view of the missing essential amino acids.

German patent DE-C 43 28 329 relates to a freeze-dried biomatrix for topical application, which comprises 10% to 80% by weight of natural polysaccharides and 20% to 90% by weight of modified polysaccharides.

EP-A-0,317,079 discloses an agent in which the components are placed into capsules practically as such, although in a compressed form, or else they are pressed into tablets. After being ingested, the capsule or the pressed tablet dissolves in the stomach, the ingredients form a swollen mass that binds liquid present in the stomach and that is broken down or eliminated via the digestive tract. Since the agent does not have any caloric value but at the same gives the body a feeling of satiation due to the swollen mass present in the stomach, food intake can then be halted or reduced without great effort for purposes of weight loss. Since the swollen mass is essentially unbound, it passes relatively quickly from the stomach to the intestinal tract, as a result of which the feeling of satiation created only lasts for a relatively short period of time.

DE 29 723 220 relates to an agent for peroral administration which contains at least one compressed, non-toxic carrier that is at least partially broken down or can be broken down, or is eliminated or can be eliminated via the digestive tract, whereby the carrier has a sponge-like structure and, at least partially, a collagen structure, after expanding in the stomach.

EP-A 0,202,159 describes a means consisting of at least one polymer that is soluble in the stomach and of at least one polymer that is not soluble in the stomach, whereby the insoluble polymer is selected from water-insoluble types of cellulose, among others.

EP-A-0,471,217 (and partially the corresponding DE-A 40 25 912, which serves as the basis for the priority) relates to an agent for oral intake with a casing that is soluble in the stomach and releases its contents; this casing is filled with a non-toxic, low-calorie substance that increases in volume upon being released, and this substance can degrade inside the digestive tract or can be eliminated through it, whereby this substance is a sponge that is placed into the casing in a compressed form and kept in this form by the casing. Said low-calorie substance is preferably a cellulose sponge or a polyurethane foam. This above-mentioned method, however, is not very feasible in actual practice since the use of a polyurethane foam as a compressed, expandable substance is approved neither in Germany nor in several other countries pursuant to the General Directive on Additives 89/107/EEC or the directives on other additives within the European Community. Moreover, the sponge cellulose or alveolar cellulose specifically mentioned for the first time in the subsequent application is not approved, at least according to food product guidelines, since these guidelines allow exclusively microcrystalline cellulose and powder cellulose, methyl cellulose, hydroxypropyl cellulose, hydroxypropylmethyl cellulose, methylethyl cellulose and sodium carboxy methyl cellulose. Also, the agent described in the above-mentioned patent application is technically complex in that one of its indispensable components is a casing, that is to say, a hard gelatin capsule, that is soluble in the stomach and that releases its contents.

The present invention has the objective of creating an agent for topical, parenteral or peroral administration. This objective is achieved by using a freeze-dried agent that comprises paramylon.

Thus, the present invention relates to an agent characterized in that it comprises 0.1% to 100% by weight, preferably 3% to 100% by weight, especially 5% to 100% by weight, of freeze-dried paramylon.

The above-mentioned freeze-dried paramylon is obtained in a per-se known manner. Reference is hereby made, for instance, to E. Ziegler in “Die natürlichen und künstlichen Aromen” [Natural and artificial aromas], Heidelberg, Germany, 1982, Chapter 4.3 “Gefriertrocken” [Freeze-drying] and the literature sources cited there as well as DE 43 28 329.

Paramylon as defined in the present invention refers to the following versions: firstly, a water-insoluble paramylon in granular form of the kind initially formed during production, having a density ranging from 1.2 g/mL to 1.7 g/mL. It also refers to a paramylon in swollen form which is obtained from granular paramylon according to the method described above, for example, by means of alkali treatment in an aqueous environment. This paramylon has a water content ranging from 94% to 99.9% by weight, that is to say, 0.1 g to 6 g of paramylon and 94 g to 99.9 g of water. The consistency of the gel thus obtained ranges from low-viscosity to almost solid, no longer flowable. It also relates to a solubilized or soluble paramylon or its derivatives, for example, ethers or esters such as carboxymethyl paramylon or paramylon sulfate. These are obtained from the swollen paramylon through hydrolysis, through enzymatic breakdown, through physical processes or through chemical reaction. At 20° C. [68° F.], this product generally has a solubility in water of 30 g to 90 g per 100 mL of water, preferably 50 g to 75 g per 100 mL of water. Finally, it pertains to a paramylon in dried form which is obtained from solubilized paramylon in a known manner and which exhibits a residual water content ranging from 1% to 15% by weight (determined after 3 hours of incubation of a 1-gram sample of the air-dried paramylon at 100° C. [212° F.] in a drying cabinet and differential weighing).

The above-mentioned paramylon is obtained through cultivation of Euglena cells in a culture medium, separation of the Euglena cells from the culture medium, isolation of the paramylon from the Euglena cells, purification of the paramylon, optionally with the addition of modified polysaccharides as well as, optionally, of biologically active ingredients and cooling, followed by freeze-drying.

As set forth in the present invention, all kinds of Euglena cells can be used such as, for instance, Euglena gracilis, Euglena intermedia, Euglena piride and other euglenoids, for example, Astaia longa. Preferred is the use of Euglena gracilis.

In this context, preference is given to isolating the paramylon from the Euglena cells without the addition of organic solvents but with the addition of at least one surfactant. Alternatively, the isolation can also be carried out without solvents or surfactants.

Even though the above-mentioned method permits the use of all algae of the Euglena class, as elaborated upon in detail in the above-referenced EP-B 417,254, preference is given especially to the use of Euglena gracilis cells as the algae.

It is also preferred for the carrier employed for the agent according to the invention to be obtained by cultivating the Euglena cells as fed-batch cultivation.

According to another preferred embodiment, the paramylon employed for later use as a carrier is isolated from the Euglena cells by means of a largely bio-degradable surfactant selected from among non-ionic or anionic surfactants.

Examples of anionic surfactants are sulfonates, such as alkyl benzene sulfonate, alkane sulfonates, α-olefin sulfonates, sulfo fatty-acid esters, sulfo succinic acid esters, sulfosuccinamates, acyloxy alkane sulfonates, acylaminoalkane sulfonates, sulfates, carboxylates, alkyl ether sulfates, alkyl aryl ether sulfates and alkyl ether carboxylates.

Examples of non-ionic surfactants are polyglycol ethers such as alkyl polyglycol ether, alkyl aryl polyglycol ether, acyl amide polyglycol ether, alkylamine polyglycol ether, ethylene oxide-propylene oxide adducts such as alkyl-ethylene oxide-propylene oxide adducts, polypropylene oxide-polyethylene oxide adducts, trifunctional ethylene oxide-propylene oxide adducts, tetrafunctional ethylene oxide-propylene oxide adducts, polyol esters such as, for instance, sugar esters, which are also known by the designation alkyl polyglycoside, polyol polyglycol ether, fatty-acid alkanol amides, fatty-acid monoethanol amides, fatty-acid diethanol amides and amine oxides.

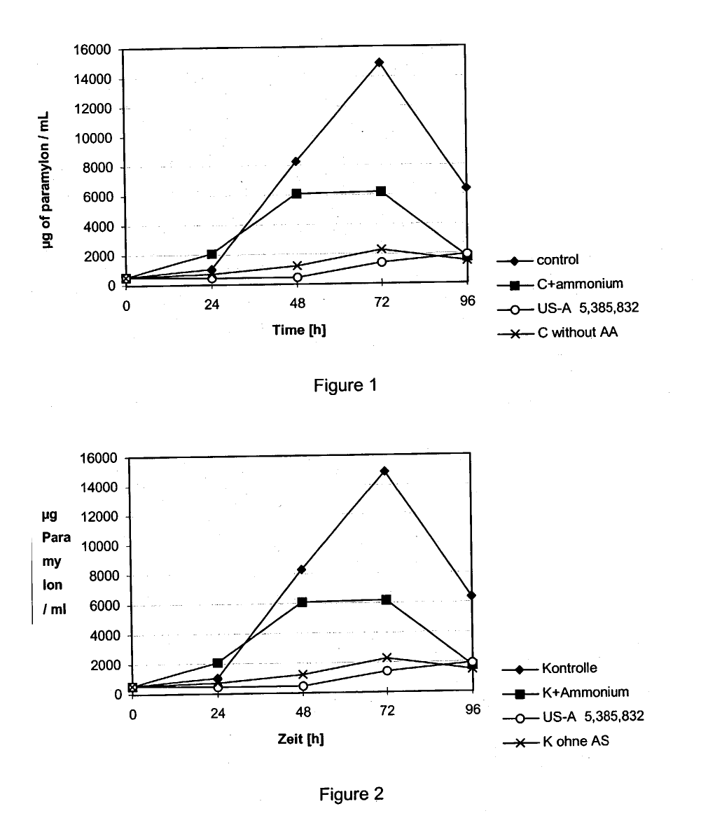

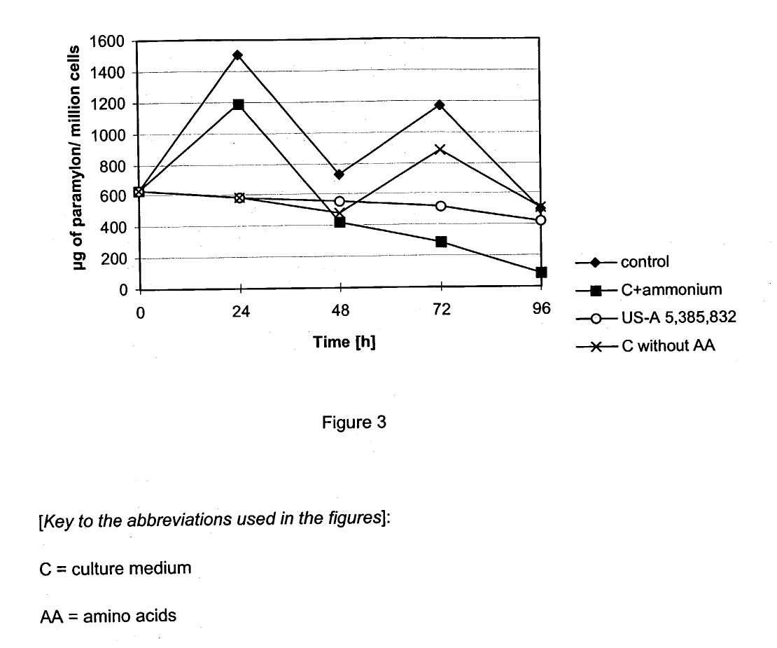

In its example of an embodiment, U.S. Pat. No. 5,385,832 describes a culture medium that makes use of ammonium sulfate as the source of nitrogen, namely, in concentrations of more than 1.9 grams per liter. This corresponds to an ammonium concentration of more than 600 mg/L, which already severely affects both the growth of Euglena and the paramylon synthesis. The medium is lacking the amino acids, some of which are essential, as was demonstrated according to the invention. These are the amino acids aspartate, glutamate and glycine. If one of these amino acids is missing, then both the growth of Euglena and the paramylon synthesis are inhibited. Moreover, it is a known phenomenon that, under ammonium stress, Euglena once again breaks down the paramylon that had been formed. This was already demonstrated by S. Sumiida et al. in “Plant and Cell Physiology” 28 (8) 1987, page 1587, especially FIG. 1 and FIG. 3. Consequently, there is no ammonium sulfate in the culture medium employed according to the invention for the production of paramylon. The ammonium concentration in the partial process step according to the invention at the beginning of the fermentation usually lies within the range from 100 mg/L to 250 mg/L and this stems exclusively from the breakdown of amino acids and from the ammonium iron-(II)-sulfate added.

The composition of the medium used according to the invention is listed below:

| | | |
|---|---|---|
| vitamin B₁ (thiamin) | 0.3 mg | to 1.0 mg |
| vitamin B₁₂ | 0.01 mg | to 0.1 mg |
| KH₂PO₄ | 0.2 g/L | to 0.6 g/L |
| MgSO₄ × 7 H₂O | 0.05 g/L | to 0.3 g/L |
| Fe(SO₄)₂(NH₄)₂ × 6 H₂O | 10 mg/L | to 30 mg/L |
| ZnSO₄ × 7 H₂O | 5 mg/L | to 20 mg/L |
| MnSO₄ × H₂O | 2 mg/L | to 10 mg/L |
| (NH₄)₆Mo₇O₂₄ × 4 H₂O | 0.5 mg/L | to 5 mg/L |
| CoSO₄ × 7 H₂O | 0.1 mg/L | to 0.5 mg/L |
| CuSO₄ × 5 H₂O | 0.3 mg/L | to 1.0 mg/L |
| H₃BO₃ | 0.1 mg/L | to 0.5 mg/L |
| NaNO₃ × 4 H₂O | 0.1 mg/L | to 1 mg/L |
| MgCO₃ | 0.1 g/L | to 0.8 g/L |
| glucose | 5 g/L | to 15 g/L |
| urea | 0.1 g/L | to 0.6 g/L |
| L-glutamic acid | 4 g/L | to 15 g/L |
| D, L-asparaginic acid | 1 g/L | to 5 g/L |
| glycine | 2 g/L | to 6 g/L |
| D, L-malic acid | 3 g/L | to 6 g/L |
| sodium succinate × 6 H₂O | 0.05 g/L | to 0.5 g/L |
| CaCO₃ | 0.05 g/L | to 0.5 g/L |

In the growth experiments described below, this medium will be referred to as culture medium (C).

The present invention will be illustrated in greater detail by means of figures.

The following is shown:

FIG. 1—the growth behavior of Euglena gracilis in various culture media.

FIG. 2—the paramylon content in Euglena gracilis over the course of the fermentation over 96 hours in various culture media.

FIG. 3—the paramylon content in Euglena during fermentation in the four culture media, relative to the cell count of 1×10⁶.

FIGS. 1 to 3 show a comparison of the growth of Euglena gracilis and the content of paramylon during the cultivation of cells in various culture media:

1) the culture medium (C) employed according to the invention for purposes of producing paramylon;

2) the culture medium described in the example of an embodiment in U.S. patent U.S. Pat. No. 5,385,832 (+EP-B 417,254) (comparative example: U.S. patent medium);

3) the culture medium employed according to the invention for purposes of producing paramylon, but to which ammonium sulfate has also been added in order to raise the ammonium concentration to 600-700 mg/L (C+ammonium);

4) the culture medium employed according to the invention for purposes of producing paramylon, but one in which the amino acids (AA) are missing (C without AA).

FIG. 1 clearly shows that the cells in the U.S. patent medium do not exceed a cell count of 2.5×10⁶/mL over the course of 96 hours. The same holds true for the medium in which the amino acids are missing. The cells in the U.S. patent medium are also highly vacuolated, which is a sign of deficiency and of the attempt to “dispose of” the ammonium. The cells contain hardly any paramylon granules. The control culture reached cell counts of about 14×10⁶/ mL, whereby the culture in the described experiment was not supplemented with nutrients. The ammonium additionally added to the control medium, however, leads to an obvious increase in the growth of the cells. This means, however, that the energy is invested primarily in cell division and not in paramylon synthesis, as is clearly evident from FIG. 2 below.

According to FIG. 2, owing to the high concentration of ammonium, the cells break down the paramylon after 72 hours. Even after 72 hours, only a total of about 6 mg of paramylon per milliliter of culture can be detected in the cells supplemented with ammonium, while about 15 mg of paramylon per milliliter of culture are to be found in the control culture. Also in the case of the control culture according to the invention, the breakdown of the paramylon sets in after 72 hours, although this happens because the carbon source has been exhausted. If, however, glucose and amino acids were to be supplemented after 72 hours, the cell count and the content of paramylon would rise again. This, however, is not the case with the ammonium culture; here, a generous amount of glucose (7 to 8 g/L) is still present in the medium after 72 hours. The control culture without amino acids as well as the US patent culture hardly synthesize any paramylon, as can be clearly seen in FIG. 1. It should still be pointed out that the cells that were cultivated in the U.S. patent medium died after about 10 to 12 days, in spite of the supply of nutrients.

FIG. 3 clearly shows that the content of paramylon in the U.S. patent culture as well as in the control medium supplemented with ammonium decreased relative to the cell count over the course of the fermentation process. The control culture as well as the control without amino acids display the fluctuations in paramylon content per cell that is caused by cell division. With every cell division, the paramylon granules naturally are distributed among the offspring cells, which leads to a temporary drop in the content of paramylon per cell. The culture without amino acids, however, synthesizes markedly lower amounts of paramylon than the control culture.

An evaluation of the figures shows that, in the cultures that have a high ammonium concentration, the paramylon is broken down, which is not desired according to the invention. Due to the absence of the amino acids that are necessary for life, hardly any cell growth can be seen in the U.S. patent cultures according to the state of the art and the cells die after some time. With the control culture, however, cell growth and the paramylon content are in an optimal ratio with respect to each other within the first 72 hours of cultivation. The cells are amply filled with paramylon. This not only allows easy isolation, but above all, ensures a higher degree of purity of the isolated paramylon, since the proportion of protein and lipid is lower relative to the paramylon quantity.

In contrast to other drying methods such as, for example, spray drying, the process of freeze-drying yields sponge-like materials that can be rapidly re-hydrated so as to have a convenience character and, due to the selection of appropriate raw materials, dimensionally stable, three-dimensional structures.

According to a preferred embodiment of the present invention, the agent comprises as the carrier 1% to 99% by weight, preferably 5% to 95% by weight, of paramylon and 1% to 99% by weight, preferably 5% to 95% by weight, of another carrier selected from among natural polysaccharides and/or modified polysaccharides and/or collagen.

According to another preferred embodiment, the agent contains as the carrier 1% to 99% by weight, preferably 5% to 95% by weight, of paramylon and 1% to 99% by weight, preferably 5% to 95% by weight, of the other carrier, namely, collagen.

Agents for Topical Administration

Therefore, the present invention relates to an agent for topical administration which comprises at least one freeze-dried carrier containing paramylon.

The above-mentioned natural polysaccharides are preferably selected from among the group consisting of pectins, alginates, carrageene, agar-agar and carob seed flour.

Examples of modified polysaccharides that can also be used as a component of the above-mentioned carrier are cellulose derivatives such as cellulose ether. Preference is given to film-forming binders such as, for instance, carboxymethyl cellulose or its derivatives. Carboxymethyl cellulose can be advantageously combined with other cellulose ethers, polyesters or polyvinyl alcohol.

The polysaccharides employed according to the invention as a carrier component can advantageously be combined with proteins of plant origin. Examples of this are soy proteins or proteins from cereals. Moreover, polysaccharides from the group of the glyco-saminoglycanes such as hyaluronic acid, its derivatives and chondroitine sulfate can be additionally employed.

In addition to the above-mentioned natural or modified polysaccharides, the agent according to the invention can contain fibers, advantageously spun fibers, for purposes of improving the stability, as well as biological active ingredients, especially cosmetic and pharmaceutical active ingredients.

According to another preferred embodiment of the present invention, it contains micelle-forming substances, for example, isoparaffins, which are typically present in an average total amount ranging from 4% to 30% by weight, especially 5% to 20% by weight.

The polysaccharides employed according to the invention as a carrier component are preferably of plant origin and, from a functional standpoint, are characterized by protective-colloidal properties. Examples of possible modified polysaccharides are all film-forming binders which, on the one hand, have a special affinity to the natural polysaccharides and, on the other hand, to the optionally employed spun fibers. The use of carboxy cellulose entails the advantage that this is a reversible water-soluble product which is non-toxic and is internationally approved as a cosmetic basic material and auxiliary (binders and thickening agents, protective colloid). Carboxymethyl cellulose also advantageously allows a combination with other cellulose ethers so that it is possible to manufacture different grades of freeze-dried biomatrices.

Basically, any natural, nature-modified or synthetic fibers can be employed in the topical agents according to the invention such as, for instance, dispersed, swollen collagen fibers. However, preference is given to spun fibers. Examples of spun fibers are cellulose ester fibers, polyester fibers, polyamine fibers, polyvinyl alcohol fibers, wool fibers, cotton fibers, silk fibers and rayon fibers, whereby rayon fibers are particularly preferred. In an advantageous manner, the spun fibers have a length ranging from 3 mm to 30 mm and a titer ranging from 1 dtex to 6 dtex (1 dtex=7.85×10⁻³ ρd²; ρ=density in g/cm³, d=diameter in μm).

In a preferred embodiment of the present invention, the freeze-dried agent containing paramylon comprises 3% to 30% by weight, especially 3% to 15% by weight, of spun fibers.

The preferred use of rayon fibers is based on the fact that these are the most hydro-philic of the normally available fibers, which is why they are best suited for making soluble products. Like the other preferred components of the carrier, the rayon fibers in the agent according to the invention are non-toxic modified polysaccharides. For this reason, they have a very great affinity to the other components, as a result of which just a small amount of these spun fibers already contributes to stabilizing the carrier. Rayon, whose use is preferred, is approved both for cosmetic and for medicinal purposes or applications.

The above-mentioned components employed as carrier materials in the agent according to the invention have themselves a skin-moisturizing effect and consequently are particularly well-suited for use in cosmetics for body care. They can also be used as a carrier material for skin-active substances whose penetration into the Stratum corneum only becomes possible, or is promoted in the desired form, due to the skin-moisturizing effect of the polysaccharides.

Examples of cosmetically active substances are numerous products such as, for example, vitamins, proteins, water-soluble plant extracts and others. The freeze-dried agents according to the invention are advantageously suitable as special body care agents for the face and skin, whereby they are particularly preferred for use as face masks.

It is possible to promote a fast percutaneous transportation of the skin-active ingredients—which takes place proportionally to the degree at which the Stratum corneum is moisturized—by using the freeze-dried agent containing paramylon, whose main components themselves have a skin-moisturizing effect. Here, small molecules that are added to the carrier or to the aqueous solution diffuse out of the concentrated area of the agent oversaturated with water and move into the less concentrated area of the epidermis. Substances like short-chain peptides, ATP, urea and electrolytes are examples of molecules that are relatively capable of penetration.

The above-mentioned ideal percutaneous transportation conditions, on the other hand, also apply to those low-molecular substances whose penetration through the horny layer of the skin is undesired and even risky because they can trigger irritation of the skin. Examples of undesired substances with proven irritation potential are known cosmetic preservatives and perfuming agents as well as surface-active substances of the type normally used in commercially available emulsion products.

The freeze-dried agents containing paramylon according to the invention are characterized by the fact that it is possible to largely and preferably completely possible to dispense with the use of the above-mentioned undesired substances.

In contrast to the familiar xerogels with which the spatial arrangement of the network (matrix) after the water removal changes to such an extent that the distances between the structural elements only reach the order of magnitude of internuclear distances, the freeze-dried agents containing paramylon according to the invention form hollow spaces into which the solvent liquid can penetrate very rapidly without hindrance. The freeze-drying method—similar to removal of water by means of sublimation from food raw materials such as, for example, tea or coffee—results in capillary structures that can be re-hydrated very rapidly. Through the use of substances with many polar groups, for instance, cellulose and proteins, the aqueous solvent tends towards considerable bridge formation between these groups, as a result of which the dissolution and diffusion behaviors are influenced. By adding sufficient water going beyond the formation of bridge bonds, the polymer matrix is made to swell, thus ensuring great mobility of the water atoms and of the active ingredients contained therein. This effect, which is observed with the freeze-dried agents according to the invention, is of paramount significance when it comes to cosmetic applications and to the desorption and penetration behavior of the cosmetic active ingredients. The freeze-dried agents according to the invention also make it possible to form moisture-stable matrix forms if the water added as a solvent comprises calcium ions in a quantity that is sufficient to partially or totally exchange sodium ions, for instance, from alginic acid salts. The spontaneously formed calcium-alginate skeleton stabilizes the agent according to the invention to such an extent that only a predefined portion of the polymers can be converted into the gel state.

Further stabilization of the freeze-dried matrix can be achieved by adding hydrophilic fiber material, for example, rayon fibers. The addition of collagen fibers accounts for dimensional stability, even after moistening. This facilitates the handling of the matrix, for instance, for skin care, in other words, topical modeling, positioning corrections, etc., since the dry stability is greatly improved.

The freeze-dried agents according to the present invention, which do not contain any spun fibers and were made of solubilized paramylon, are characterized by the fact that the gel created for cosmetic application can be massaged into the skin until it completely disappears, whereas agents containing structural fibers always leave insoluble fiber residues on the skin which then have to be removed after the cosmetic treatment.

In any case, as a result of the selective use of fibers or of stabilization with calcium ions, the consistency of the freeze-dried agents according to the invention can be systematically adjusted, thus allowing an adaptation of the product to the desired application area, a process which ensures the requisite simple and practical handling.

According to another preferred embodiment, substances capable of micelle formation are added to the freeze-dried agent containing paramylon according to the invention. These substances are present in the freeze-dried agents according to the invention in amounts ranging from 4% to 30% by weight, preferably from 5% to 20% by weight. With these biomatrices, the micelle-forming substances used are, for example, isoparaffins which, as a result of micelle formation, can build up a coherent skeleton that allows the production of stable gels.

Another preferred aspect is that the agents according to the invention do not contain any perfuming agents, dyes or preservatives.

Cosmetic and/or plant-based and/or pharmaceutical active ingredients, preferably in an amount ranging from 0.1% to 50% by weight, and especially from 3% to 30% by weight, can also be easily incorporated into the freeze-dried, topical substances according to the invention. It is an advantageous aspect to incorporate the active ingredients in an encapsulated form such as, for example, in liposomal or liposome-like vesicles.

In comparison to known biopolymer sponges, the freeze-dried agents according to the invention have the advantages described below.

It is possible to completely dispense with the use of skin irritants substances such as, for instance, preservatives, dyes and perfuming agents. The topical agents according to the invention are also particularly well-suited for the manufacture of galenic systems which can effectuate a much more controlled and targeted release of the active ingredients onto the skin than is the case with the galenic systems known from state of the art. Thus, with the freeze-dried agents according to the invention, active ingredients can be dosed with precision within narrow tolerance ranges and at a high activity potential, for example, in the case of vitamin A derivatives. The activity potentials of conventional active ingredients are achieved due to unhindered transportation routes through the skin, due to the absence of interactions, due to shorter transportation routes, as a result of the lack of barrier substances, for instance, fats, and also in view of higher possible dosing levels, that is to say, greater active concentration. In this manner, the agents according to the invention translate into a more economical utilization of the active ingredients.

Manufacture of Agents for Topical Administration

The freeze-dried agents according to the invention are manufactured by first producing so-called swollen paramylon. For this purpose, the granular or crystalline paramylon is first dissolved in an NaOH solution, then neutralized with HCl, whereby paramylon fibrils are precipitated as a gelatinous mucilage. Undesired salts are removed by washing the mucilage several times. The viscosity and dry weight levels of the swollen paramylon can be adjusted by adding water. Or else, crystalline paramylon is solubilized analogously to the state of the art—U.S. Pat. No. 5,663,324—for zymosan obtained from yeast (16.9% to 22.3% mannan, 50.7% to 57.8% β-1,6-glucan β-1,3-glucan chains and protein, amylases and lipids as the rest; see “Polysaccharide, die immunstimulierend wirken” [Polysaccharides having an immuno-stimulating effect] by Hansel R. 1987, in Farmaceutisch Tijdschrift voor België [Belgian pharmaceutical journal] 64, pages 313-326). In addition to swollen paramylon or to solubilized paramylon, the other carriers—selected from among natural polysaccharides and/or modified polysaccharides and/or collagen as well as optionally the desired cosmetically or pharmaceutically active substances—are uniformly mixed together in an aqueous medium, after which the mixture is cooled. A gel is formed during the cooling process. Subsequently, the spun fibers are gently introduced into this gel and uniformly distributed. Following agitation and renewed cooling, for example, down to about 1° C. [33.8° F.], the compound is poured into molds. The original gel structure forms again in these molds and the subsequent freeze-drying procedure yields a material that is structurally very similar to a pure collagen sponge. As the first phase of formation of the later matrix, freezing is an essential process step whereby, according to the invention, preference is given to accelerated freezing at low temperatures. As a result of the subsequent freeze-drying of the freeze-dried gel in a high vacuum, the solvent is frozen out and condensed (sublimation). An essential characteristic of freeze drying is pore formation without volume change. The effect of rapid re-hydration is based on this.

In a preferred embodiment of the present invention, first a mixture consisting of the swollen paramylon and of the other carrier components is prepared, which is then stirred into water. After the mixture has been cooled to 10° C. [50° F.], a mixture consisting of spun fibers, cosmetic and/or pharmaceutical active ingredients and/or micelle-forming substances can be dispersed into this premix. The resultant mixture is frozen in the form of plates at −10° C. to −60° C. [14° F. to −76° F.], preferably at about −20° C. [−4° F.], over a time period of 0.5 to 4 hours, preferably 1 to 3 hours. Plates having a layer thickness of 0.5 cm to 5 cm, preferably 1.5 cm to 2.5 cm, are preferred. During freezing, the pore size is essentially controlled by the freezing speed and the temperature characteristics. Optionally, the plates can be put into intermediate storage at 10° C. to −50° C. [14° F. to −58° F.] before they are freeze-dried at a heating temperature within the range from 40° C. and 150° C. [104° F. and 302° F.] and a vacuum of about 0.5 mbar to 3.0 mbar. Preferably, the freeze-drying process should be carried out over a time span of about 15 to 48 hours. Afterwards, the plates can be split and prepared.

After the freeze-drying process, the water content of the agents containing freeze-dried carrier materials obtained according to the above-mentioned method preferably lies within the range from 5% to 15%, 10% being particularly preferred. The dry-substance concentration of the starting material employed in the production of the freeze-dried agent according to the invention, that is to say, the mixture of components in the de-mineralized water, amounts to about 1% to 5%.

Use of Agents for Topical Administration

The freeze-dried agents according to the invention can be employed for the topical transdermal administration of active ingredients; in preferred embodiments, these agents serve as plasters, for example, as an immuno-stimulant or, in an especially preferred form, as a drug delivery system. In a particularly advantageous manner, the freeze-dried agents according to the invention can be used for the topical or transdermal application of cosmetic or pharmaceutical active ingredients, in other words, as a facial treatment or a face mask.

Agents for Peroral Administration

Therefore, the present invention also pertains to agents for peroral administration which contain at least one freeze-dried carrier containing paramylon. Such products are usually administered in granular form or else compressed, for example, as a powder and optionally provided in advance with a shell and/or in encapsulated form, as will be elaborated upon in detail below.

According to a preferred embodiment of the agent orally administered, the carrier is first compressed and this carrier has a sponge-like structure once it has expanded in the stomach. Then, in order for the carrier to acquire an elastic sponge structure, in addition to 1% to 80% by weight, preferably 5% to 75% by weight, of paramylon, it also has to contain 20% to 99% by weight, preferably 25% to 95% by weight, of the other carrier component, namely, collagen.

The collagen structures used according to the invention as another carrier component essentially refer to the so-called scleroproteins which are also known as fibrous proteins, skeletal proteins or structural proteins and which constitute a group of water-insoluble, fibrous, animal proteins having a purely skeletal and supporting function. The collagen is obtained from supporting and connective tissue, skin, bone and cartilage.

In another preferred embodiment, the other carrier component, namely, collagen, in the agent according to the invention comprises the amino acids glycine and hydroxy-proline, with the tripeptide sequence GlyXy, wherein X stands for any desired amino acid and hydroxyproline often appears instead of y.

According to another preferred embodiment, the additional component in the carrier, namely, collagen, stems from the phylum Porifera, especially the class of Demospongiae. This is the zoological designation of the group of aquatic animals commonly referred to as sponges. These sea inhabitants have a form that is without symmetry but organized in a polar manner as clusters, crusts, funnels and bowls, and as mushrooms and antlers, that is made up of a skeleton consisting of collagen-(spongin) fibers in which scleres of calcite or silicic acid are deposited. The sponges normally have three layers, of which the largest middle layer, namely, the mesohyl, consists of a gelatinous matrix containing collagen fibers. Please refer, for example, to “Lexikon der Biologie” [Encyclopedia of biology], volume 7, Freiburg, Germany 1986, under the entry “sponges” as well as op. cit. volume 8 under the entries “spongia”, “spongin”.

The phylum Porifera is divided into the classes Calcarea, that is to say, sponges with calcite deposits, Hexactinellida, in other words, those with special silicic acid deposits as well as Demospongiae, which encompasses those with a skeleton consisting of fiber or silicic acid. The preferred class Demospongiae includes, in particular, the horn siliceous sponge (Cornacu-spongia), the freshwater sponges and the bath sponge (Spongia officinalis) with the subspecies Levantine sponge (Spongia officinalis mollissima), zimocca sponge (Spongia officinalis zimocca), elephant-ear sponge (Spongia officinalis lamella) as well as the horse sponge (Hippospongia communis) with its large holes.

The sponges harvested from the water are freed of mineral components in a known manner, for instance, by means of acidic digestion, so as to make it possible to isolate the collagen carrier as the essential component of the agent according to the invention.

According to another preferred embodiment, the additional carrier, namely, collagen, in the agent employed according to the invention is a collagen derived from natural animal substances. The production of these collagen fiber networks or collagen sponges whose use is preferred —is a familiar process known, for example, from German laid-open application no. 18 11 290, German laid-open application no. 26 25 289, German patent no. 27 34 503 and especially from German laid-open application no. 32 03 957 of the applicant.

In accordance with another preferred embodiment, after the compressing procedure, the agent according to the invention has a sponge-like carrier with a density ranging from 0.005 g/cm³ to 1.0 g/cm³, preferably from 0.01 glcm³ to 0.1 g/cm³. The density cited is measured according to German standard DIN 53 420.

In another preferred embodiment, the carrier in the agent according to the invention is not encapsulated, but rather, it is in the form of a pressed blank. In this context, we would like to refer to the monograph by Ms. Schöffling-Krause titled “Arzneimittelformenlehre” [Treatise on drug delivery systems], Stuttgart, Germany, third edition, 1998, pages 181 through 210 and to the production methods and machines described there as well as to the chapter titled “Tablets” in the monograph by Rudolf Voigt titled “Pharmazeutische Technologie fur Studium und Beruf” [Pharmaceutical technology for students and professionals], published by Ullstein Mosby, Berlin, Germany 1993, page 205 ff. as well as to the production methods and machines described there. The material feed to the tablet presses is modified as a function of the material.

According to another preferred embodiment, the carrier in the agent according to the invention is in the form of a tablet. Once again, we would like to refer to the monograph by Schöffling-Krause. Depending on the production conditions, this tablet comprises 0.001 grams to 5 grams, preferably 0.2 grams to 1 gram, relative to 100 grams of the agent, of at least one lubricant in the form of a (matrix) mold-release agent. Examples of this are siliconized talcum, cetyl talcum, magnesium stearate, PEG 4000-6000, stearic acid, cetyl alcohol, paraffin, beeswax, hydrated fats and oils and other physiologically tolerable mold-release agents. An overview of this can be found in the monograph by Rudolf Voigt in the chapter titled “Tablets”. Here, special preference is given to the use of an oblong tablet.

In another preferred embodiment, the tablet has a soluble coating covering the tablet. In this context, reference is made, for example, to the monograph by Schöffling-Krause, 1998, pages 93 through 98 as well as to Bauer, pages 397 through 413. This coating is normally applied in amounts of 0.1 grams to 50 grams, preferably from 1 gram to 20 grams, relative to 100 grams of the agent, and can consist, for instance, of film-forming coatings that are soluble in gastric juice such as, for example, a coating syrup on the basis of hydrogels or coating powders, color pigment suspensions, a smooth syrup or a hard wax solution or suspension. Film coatings with polymers that are resistant to saliva but that are soluble in gastric juice such as, for instance, polyacrylates, are also employed. Other film coatings are soluble cellulose derivatives such as hydroxypropyl cellulose. An overview of suitable film-forming substances is found, once again, in the monograph by Voigt in the chapter titled “Dragées” on page 261 ff. Other suitable coatings are those produced according to the method used for making sugar dragées, as can be likewise seen in the chapter titled “Dragées” in the monograph by Voigt.

According to another preferred embodiment, the carrier in the agent according to the invention is encapsulated, that is to say, it is contained in a capsule that is soluble in gastric juice, for example, in the form of a soft-gelatin capsule, a gelatin hard-shell capsule or as a capsule with a modified release of the active ingredient. In this context, we would like to refer to the monograph by Schöffling-Krause titled “Arzneimittelformenlehre” [Treatise on drug delivery systems], Stuttgart, Germany, 1998, pages 64 through 81 and to the production methods and machines described there as well as to the chapter titled “Capsules” in the monograph by Rudolf Voigt titled “Pharmazeutische Technologie für Studium und Beruf” [Pharmaceutical technology for students and professionals], published by Ullstein Mosby, Berlin, Germany 1993, and to the production methods and machines described there.

According to another preferred embodiment, the carrier of the agent according to the invention comprises at least one active ingredient and/or additive. The active ingredients are added at various points in time during the manufacture of the sponge-like carrier materials. Examples of additives are approved colorants such as carotenoids or vitamins such as for instance, vitamin B₂. Active ingredients such as, for example, omeprazol can also be added at various points in time, for instance, prior to compressing the sponges.

According to another preferred embodiment of the agent according to the invention, the active ingredient is contained in a matrix, casing, bedding and/or another carrier material that controls the release. This effectuates the release of the active ingredient by means of membrane diffusion, pore diffusion, swelling, erosion, pore diffusion from the matrix, swelling with diffusion as well as swelling with disintegration. Here, reference is made to the monograph by R. Voigt, the chapter titled “Perorale Depotarzneimittelformen” [Peroral depot drug delivery system] as well as to Bauer, pages 533 to 555 and Schöffling, 1998, page 176 ff. and pages 199 to 205. In particular, hydroxypropyl methyl cellulose is employed in this case as the carrier material that controls the release.

Production of Agents for Peroral Administration

The present invention also has the objective of providing a process for the production of the above-mentioned agent.

Therefore, the invention also relates to a process for the production of the above-mentioned agent, characterized in that a fine-pore, freeze-dried sponge having a density ranging from 0.0005 g/cm³ to 1.0 g/cm³—which has optionally been treated with at least one active ingredient and/or additive prior to the compressing procedure and optionally also with the use of a mold-release agent—is compressed to one-half to one-fiftieth, preferably one-third to one-thirtieth of its original size and optionally surrounded by a capsule that is soluble in gastric juice.

In another preferred embodiment of the process according to the invention, the fine-pore sponge is combined with a carrier layer for at least one active ingredient. In accordance with the production process for layered tablets, the carrier layer is compressed onto the pre-compacted sponge.

According to another preferred embodiment of the process according to the invention, the fine-pore sponge is treated with at least one active ingredient and/or additive before or during the compressing procedure, which consists at least of one step. This is preferably done in that the active ingredients and/or additives are applied in a familiar manner onto the carrier in the form of the sponge, for instance, either in pure form, dissolved in a solvent or else as a dispersion in the form of an emulsion or suspension.

The production and compression of the sponges are done, for example, after pre-compacting the sponge once it has been placed into an eccentric press and using a compression tool with a lower and upper punch commonly employed for tablet production and with a suitable matrix (for instance, an oblong form, 1.8 cm×0.9 cm). With the punching, a pre-compacted sponge is compressed to form a tablet having a thickness of 4 mm. Active ingredients can also be incorporated into the collagen dispersion prior to the freeze-drying process.

Use of Agents for Peroral Administration

Within the context of the present invention, a biologically active substance such as, for instance, a cosmetic or a pharmaceutical, can be employed as the active ingredient which, in particular, can be released during the time of residence in the stomach.

In addition to this, minerals and trace elements can also be employed as such active ingredients.

This is preferably done in that nutritional supplements, particularly vitamins, minerals, fatty acids and/or dietary fiber are also added or incorporated into these carriers.

Examples of such nutritional supplements are vitamins, which are known to be divided into fat-soluble vitamins such as, for instance, retinol, retinoic acid, retinal, calciferol, that is to say, the D vitamins, the tocopherols or E vitamins and the K vitamins or phylloquinones. Vitamin A deficiency causes night blindness, vitamin D deficiency causes rickets and vitamin E deficiency increases the tendency towards oxidative hemolysis, causes hemolytic anemia, edema and increased irritability. Vitamin K deficiency impairs blood clotting and causes hemorrhaging.

Another group that can be employed according to the invention in the nutritional supplements includes water-soluble vitamins, such as vitamins of the B group, for example, vitamin B₁, thiamin, riboflavin, pyroxidine, nicotinic acid, corrinoids, folic acid and, as another group, ascorbic acid or vitamin C. Thiamin deficiency leads to beriberi, riboflavin deficiency can cause inflammation of the cornea and gives rise to increased vascularization. B₆-vitamin deficiency can cause seborrheic dermatitis, hypochromic anemia, peripheral neuritides as well as cerebral convulsions. There is an increased need for vitamin B₆ during pregnancy and following radiation therapy. A deficiency of nicotinic acid leads to pellagra, while a shortage of corrinoids causes pernicious anemia or even funicular myelosis. Deficiency of folic acid causes problems during pregnancy. Insufficient ascorbic acid leads to scurvy and to Möller-Barlow's disease.

The daily intake of vitamins via the agents according to the invention ensues, for example, from the recommendation for supplement intake as put forward by the German Society for Nutrition (DGE). Typical daily intakes of vitamins are also cited, for instance, in the monograph by Forth titled “Pharmakologie und Toxikologie” [Pharmacology and toxicology], 4^{th} edition, 1983, page 401.

Other typical components of the oral agent according to the invention used as a nutritional supplement can be minerals or trace elements which are to be supplied for prophylactic or therapeutic purposes. Examples of these are iron, zinc, copper, manganese, molybdenum, iodine, cobalt and selenium as essential elements for the human body. When it comes to the typical daily requirement, reference is made to the above-mentioned monograph by Forth, the table on page 416.

In addition to the essential elements for the human body, in many cases it is also necessary to supplement calcium, which is not only needed for the bones and cell structure, but also for the entire metabolism of the body. The quantity of calcium normally obtained by the body from food is not always sufficient to meet the needs. Calcium provides bones and teeth with their strength.

Another essential element that can be supplied according to the invention is potassium, which plays an active role in the regulation of the osmotic pressure within the cells. Potassium is a component of the digestive tract of the stomach and intestines and is quickly resorbed.

Another essential component for nutritional supplementation is magnesium, which influences muscle function. Magnesium is an essential nutrient which is present in almost all cells and which controls the activation of enzymes involved in energy metabolism.

In addition, the agents according to the invention can also be employed to administer at least one, at least partially soluble, pharmacologically active substance, especially one with a local or systemic effect. This includes, for instance, pharmacologically active substances that act upon the central nervous system such as, for example, depressants, hypnotics, sedatives, tranquilizers, muscle relaxants, antiparkinsonian drugs, analgesics, antihypertensive drugs, chemotherapeutic agents, antiinflammatories, hormones, contraceptives, sympathomimetics, diuretics, antiparasitic agents, agents for the treatment of hyperglycemia, electrolytes, cardiovascular drugs.

Examples of water-soluble pharmaceuticals which can be delivered with a delayed release by the agent according to the invention include iron sulfate, aminocaproic acid, potassium chloride, mecamylamine hydrochloride, procaine hydrochloride, amphetamine sulfate, methamphetamine hydrochloride, phenmetrazine hydrochloride, bethanechol chloride, atropine sulfate, methascopolamine bromide, isopropamidiodide, tridihexethyl chloride, oxoprenolone hydrochloride, metroprolone hydrochloride, cimetidine hydrochloride and the like.

Examples of pharmacologically active substances with limited solubility in water which can be released by the agent according to the invention are mecitine hydrochloride, phenoxy benzamine, thiethyl perazine maleate, anisindone, reserpine, acetolamide, methazol amide, chloropropamide, tolazimide, chloromadinone acetate, aspirin, progestin, corticosteroids, etc. When it comes to examples of medicinal drugs that can be released by the agent according to the invention, reference is made to the “Pharmazeutische Stoffliste” [Pharmaceutical substance list], 7^{th} edition, Frankfurt am Main, Germany, 1989. Typical examples of medicinal drugs that can be incorporated into such carriers are acyclovir, levodopa and riboflavin.

According to another preferred embodiment of the agent according to the invention, the latter is employed in a form that has a time of residence in the stomach, that is to say, it can stay in the stomach for several hours.

The present invention also relates to the use of the above-mentioned agent for purposes of modified active-ingredient release.

The agent according to the invention for oral administration is particularly well-suited for the production of a drug for the therapeutic or prophylactic treatment of diseases of the digestive tract, especially stomach diseases such as endogastritis, or for the prophylaxis and treatment of diseases for which a stimulation of the immune system is called for.

Agents for Parenteral Administration

The present invention also relates to an agent for parenteral administration comprising at least one freeze-dried carrier containing paramylon. In particular, these are depot implants or depot parenteral systems by means of which the biological, especially pharmaceutical active ingredients can be administered over the course of days, weeks or even months once the implant, for example, has been administered subcutaneously or intramuscularly. These implants normally have a thickness within the range from 0.5 mm to 5 mm, preferably 1 mm to 4 mm and, at a weight of, for instance, 50 mg, they can release up to 10 mg of pharmaceutical active ingredient per month.

Finally, the present invention also relates to an agent containing 1% to 80% by weight, preferably 5% to 75% by weight, of paramylon and 20% to 99% by weight, preferably 75% to 95% by weight, of the additional carrier, namely, collagen, in the form of a freeze-dried three-dimensional biomatrix, for the topical and/or parenteral administration and/or application of paramylon via body openings. Such an agent or galenic embodiment is also dimensionally stable over a long period of time, even after being moistened or in the presence of body fluids, it can be resorbed by the body and it is capable of supplying the body with pharmacologically effective quantities of paramylon both topically via the body surface as well as parenterally, for instance, subcutaneously and it can also be applied via body openings, for example, orally, nasally, vaginally or rectally.

The present invention will be explained below with reference to production and application examples and will also be compared to the state of the art. In this context, the term “parts” always refers to parts by weight.

A distinction between the glucan (zymosan) obtained from yeast and the paramylon obtained from algae can be made as follows through enzymatic breakdown due to the presence or absence of glucose. If the paramylon is broken down with a specific enzyme, namely, β-1,3-glucan (Merck, for instance, from the Roman snail—Helix pomatia), glucose is obtained that can be detected by means of thin-layer chromatography. If, in contrast, the paramylon is cleaved with a-1,4-amylases, no breakdown product is obtained. In this manner, the type of bond can be specifically confirmed. Yeast glucan is additionally 1,6-cross-linked, as a result of which it does not yield pure glucose when broken down with b-1,3-glucanase, but rather several cleavage products, also disaccharides. In order to confirm the type of bond, a few μg of paramylon (e.g. 100 μL) at a pH of 5 are mixed with 0.2 mg/mL of β-1,3-glucanase and incubated at 35° C. [95° F.] in a water bath for a maximum of 12 hours. Subsequently, the breakdown reaction is stopped by means of boiling for 2 minutes. Afterwards, 10 mL to 50 mL of the clear supernatant are applied onto a silica gel 60 plate (Merck, 10 cm×10 cm) and developed for 2.5 to 3 hours in a thin-layer chromatographic chamber with the solvent system consisting of isopropanol:glacial acetic acid:water (29:4:9). Finally, the plate is sprayed with aniline-phthalate reagent (Merck) or orcinol-sulfuric acid reagent (Merck) and incubated for 5 minutes at 100° C. [212° F.] in a drying cabinet. The glucose bands become visible in this manner.

### Production Example 1

(Freeze-dried Paramylon without Active Ingredient):

Approximately 15 to 20×10⁶ Euglena gracilis cells (available under no. 1224-5/25 (Euglena gracilis strain Z) from the strain collection for algae cultures of the Plant Physiology Institute of the University of Gottingen, Germany) underwent fed-batch cultivation within a period of time ranging from 0 to about 120 hours, typically about 72 hours. This means that the cells are not cultivated in a closed batch system with a one-time addition of nutrients at the beginning of the fermentation, but rather, nutrients were added as supplements to the system at certain time intervals. These nutrients were primarily glucose, amino acids and vitamins. In this manner, optimal growth of the cells is ensured as well as optimal metabolism of the added glucose to form paramylon. The temperature should lie within the range from 20° C. to 30° C. [68° F. to 86° F.]; this is the temperature at which Euglena gracilis brings about optimum growth rates. Moreover, an adequate oxygen supply to the cells is essential for an optimum paramylon synthesis rate. This is controlled during the entire cultivation and set above the culture medium at values between 0 and 20 N liters of air per minute through the addition of oxygen and the removal of the carbon dioxide generated. The oxygen saturation typically lies between 20% and 80%. This ensures up to 90% reaction of the added amount of glucose to form paramylon.

The yields lie within the range from 12 to 18 grams of paramylon per liter. of cell culture after a cultivation time of approximately 72 hours. The pH value during the fermentation lies within the range from 3.5 to 6 and, if necessary, is back-titrated through the addition of acid.

Subsequent to the cultivation, the cells are separated by means of centrifugation or simple sedimentation of the culture medium and re-suspended in water. Afterwards, the cells undergo lysis with ultrasound, for example, at 400 watt. The sedimented paramylon is finally washed with an anionic or non-ionic surfactant, for instance, the fatty alkyl polyglycosides Plantaren® or Glucupon® (Henkel KGaA) or with the fatty alcohol ether sulfate ZETESOL (Zschimmer & Schwarz GmbH)—both of which are bio-degradable surfactants, and then treated with ultrasound. Another possibility consists of washing with an anionic surfactant, such as sodium dodecyl sulfate (SDS) or in boiling with SDS under reflux. The supernatant is subsequently discarded once the paramylon has sedimented. Subsequently, the paramylon is washed with water and can then be frozen or dried.

The paramylon obtained on the basis of the method according to the invention exhibits a purity of more than 99% in accordance with elementary analysis and residual protein determinations. When the paramylon is cold-washed with the above-mentioned surfactants, the residual protein content ranges from 0.07% to 0.09% by weight, while after being hot-washed, the residual protein content amounts to a mere 0.01% to 0.03% by weight.

After being washed, the paramylon is dissolved under agitation in 0.5 to 1 M aqueous sodium hydroxide, resulting in a paramylon concentration of approximately 5% to 10% in sodium hydroxide solution. Subsequently, the solution is diluted with water so as to yield 5 to 10 times the original volume and neutralized with concentrated HCl or with 1 M HCl. The resultant gelatinous compound is subsequently washed with water and freed of the sodium chloride.

The addition of water finally produces a pourable 0.5% to 1%-compound that is then poured on a smooth surface to form layers having a thickness of about 0.5 to 3 cm, which are then frozen at around −40° C. [−40° F.]. Subsequently, the layers are freeze-dried.

The biomatrices produced in this manner can be essentially characterized in terms of their density; it amounts to about 0.003 to 0.2 g/cm³ for the matrices. Such biomatrices can be employed topically or parenterally.

### Production Example 2

(Freeze-dried Paramylon with Collagen:

Production Example 1 was repeated, although, prior to the freeze-drying procedure, a mixture of 15% by weight of paramylon and 85% by weight of collagen is made by mixing, a process in which water is used as the solvent.

### Production Example 3

(Freeze-dried Paramylon with Carboxymethyl Cellulose and Commonly Employed Additives):

Production Example 1 was repeated, although, prior to the freeze-drying procedure, a mixture consisting of 85 grams of paramylon and 10 grams of carboxymethyl cellulose underwent freeze-drying using 1000 grams of de-mineralized water. The product can be used for topical purposes, for example, as a face mask.

### Production Example 4

(Agent for Peroral Administration, without Active Ingredient:

A sponge made according to Production Example 2 (length of 46 cm, width of 8 cm, thickness of 1.3 cm) weighing 12 grams is pre-compacted by means of a pneumatic press to a width of 1.5 cm, thus producing a strip (measuring 46 cm in length, 1.5 cm in width and 1.3 cm in thickness).

The strip is placed in segments into an eccentric press (tablet press EK 0, manufactured by the Korsch company of Berlin, Germany) and the material is punched out using a lower and upper punch as well as a matrix so as to form an oblong tablet (19 mm×8 mm), each with four notches on the top and bottom. The tablets have a thickness of 4 mm at a weight of approximately 200 mg. The tablets are dimensionally stable after the compressing operation. The pressed blanks expand in water at a temperature of 37° C. [98.6° F.] while absorbing liquid within a maximum of 5 minutes to form a sponge (1.9 cm×0.8 cm×8 cm). The peroral agent thus obtained did not exhibit any volume increase, even after being stored for at least 2 months in a humid atmosphere.

### Production Example 5

(Agent for Peroral Administration with Mold-release Agent)

A sponge containing paramylon is pre-compacted to form a strip, as described in Production Example 4. The top and bottom of the strip are coated with the pulverulent mold-release agent magnesium stearate prior to the compressing operation. In the pres??ent example, 65 mg of magnesium stearate are used per strip. Each tablet comprises approximately 2 mg of mold-release agent on its surface. As a result of the hydrophobic mold-release agent, the initial expansion of the sponge within the first minute is delayed. In water at a temperature of 37° C. [98.6° F.], the pressed blanks expand and absorb liquid within 5 minutes to form a sponge (1.9 cm×0.8 cm×8 cm). The peroral agent thus obtained did not exhibit any volume increase, even after being stored for at least 2 months in a humid atmosphere.

### Production Example 6

(Agent for Peroral Administration as a Nutritional Supplement)

Production Example 4 was repeated whereby, in addition, 5 grams of natural vitamin E, relative to 100 grams of the agent, were added. The nutritional supplement thus obtained did not exhibit any volume increase, even after being stored for at least 2 months in a humid atmosphere.

### Production Example 7

(Agent for Peroral Administration with a Pharmaceutical Substance)

Production Example 4 was repeated, except that, in addition, 20 grams of the drug levodopa (L-dioxyphenyl alanine), relative to 100 grams of the agent, were added to the fine-pore sponge starting material. The nutritional supplemented thus obtained did not exhibit any volume increase, even after being stored for 2 months in a humid atmosphere.

### Production Example 8

(Agent for Peroral Administration with a Modified Active-ingredient release)

Production Example 4 was repeated whereby, as a modified active-ingredient release system, the compressed sponge is combined with another layer consisting of 200 mg of hydroxypropylmethyl cellulose containing approximately 100 mg of levodopa and 25 mg of benserazide (1-DL-serine-2-(2,3,4-trihydroxybenzyl) hydrazide) as the carrier for the depot drug. The release of the active ingredient takes place in vitro within 10 hours. The peroral agent thus obtained did not exhibit any volume increase, even after being stored for at least 2 months in a humid atmosphere.

### Application Example 1

(For Oral Administration in Vitro):

The pressed blank described in Production Example 2 was tested at 37° C. [98.6° F.] for its degradability in the digestive tract by being placed in synthetic gastric juice according to the US Pharmacopoeia (USP XXIII) with the addition of pepsin. This experiment indicated that the sponge began to break down after 240 minutes. After the gastric juice was replaced with synthetic intestinal juice according to the US Pharmacopoeia (USP XXIII) with the addition of pancreatin, the sponges disintegrated completely after about 5 hours. Also when placed into synthetic intestinal juice, the sponge dissolved completely within about 7 hours.

### Application Example 2

(For Topical Administration in Vitro):

A sponge containing paramylon according to Production Example 2 was tested at 37° C. [98.6° F.] for its resistance to electrolyte in a physiological saline solution (0.09% by weight of NaCl) as a model for a lymphatic fluid. A visual examination did not reveal any changes such as, for instance, breakdown, within 3 days. In contrast, a sponge made of pure paramylon according to Production Example 1 already disintegrated after 180 minutes.

1. An agent, characterized in that it comprises 0.1% to 100% by weight, preferably 3% to 100% by weight, especially 5% to 100% by weight, of freeze-dried paramylon.

2. The agent according to claim 1, characterized in that it comprises 0.1% to 99% by weight, preferably 5% to 95% by weight, of paramylon and 1% to 99% by weight, preferably 5% to 95% by weight, of another carrier selected from among natural polysaccharides and/or modified polysaccharides and/or collagen.

3. The agent according to claim 1, characterized in that it comprises 0.1% to 99% by weight, preferably 5% to 95% by weight, of paramylon and 1% to 99% by weight, preferably 5% to 95% by weight, of the other carrier, namely, collagen.

4. The agent according to claims 1 to 3, characterized in that it serves for the local and/or topical application of biologically active ingredients and in that it can be obtained by reacting an aqueous mixture of the above-mentioned carrier(s) and, optionally, of biologically active ingredients, by cooling and by subsequent freeze-drying of the resultant gel.

5. The agent according to claims 1 to 4, characterized in that it also comprises fibers, calcium ions, cosmetically and pharmaceutically active ingredients and micelleforming substances, which are added prior to the freeze-drying procedure.

6. The agent according to the claim 1, characterized in that it comprises 25% to 75% by weight of paramylon and 25% to 70% by weight of modified polysaccharides as well as 5% to 15% by weight of water.

7. The agent according to the preceding claims, characterized in that it comprises film-forming binders as the modified polysaccharides.

8. The agent according to the preceding claims, characterized in that it comprises cellulose-ether derivatives or cellulose-ester derivatives as the modified polysaccharides.

9. The agent according to the claim 8, characterized in that it additionally comprises cellulose ester, polyester or polyvinyl alcohol as fibers in the form of spun fibers.

10. The agent according to the claims 1 to 9, characterized in that it comprises 3% to 30% by weight, preferably 3% to 15% by weight, of spun fibers.

11. The agent according to one of claims 8 to 10, characterized in that the spun fibers are selected from the group consisting of cellulose esters, polyesters, polyamide, polyvinyl alcohol, wool, cotton, silk and rayon fibers.

12. The agent according to one of claims 5 or 9 to 11, characterized in that the spun fibers have a length of 3 to 30 mm and a titer of 1 to 6 dtex.

13. The agent according to claims 1 to 12, characterized in that it comprises additional plant-based and/or animal proteins.

14. The agent according to claims 1 to 13, characterized in that it additionally comprises glucosaminoglucanes.

15. The agent according to claim 14, characterized in that the glucosaminoglucanes are selected from among the group consisting of hyaluronic acid, its derivatives and/or chondroitine sulfate.

16. The agent according to claims 1 to 15, characterized in that it comprises micelle-forming substances.

17. The agent according to claim 16, characterized in that the micelle-forming substances are isoparaffins.

18. The agent according to claims 1 to 3, characterized in that the carrier comprises cosmetic and/or pharmaceutical active ingredients as the biologically active ingredients.

19. The agent according to claim 18, characterized in that it comprises the active ingredients in an amount ranging from 0.1% to 50% by weight, especially 3% to 30% by weight.

20. The agent according to one of claims 18 or 19, characterized in that the active ingredients are present in encapsulated form.

21. The agent according to claim 20, characterized in that the active ingredients are encapsulated in liposomal or liposome-like vesicles.

22. The agent according to one of claims 1 to 21, characterized in that the agent is stabilized against moisture by means of calcium ions.

23. Use of the agent according to claims 3 to 22 as a plaster, especially as a drug delivery system or a component of transdermal patches.

24. Use of a freeze-dried agent according to claims 1 to 22 as a facial treatment or face mask.

25. A method to produce the freeze-dried paramylon carrier according to claims 1 to 22, by means of the
cultivation of Euglena cells in a culture medium,
separation of the Euglena cells from the culture medium,
isolation of the paramylon from the Euglena cells,
purification of the paramylon,
reaction of the paramylon and/or of the modified paramylon, of the optionally modified polysaccharides and other carriers according to claim 2 and, if applicable, of the biologically active ingredients,
cooling and subsequent freeze-drying.

26. The method according to claim 25, characterized in that the paramylon is isolated from the Euglena cells without the addition of organic solvent but with/without the addition of a surfactant.

27. The method according to claim 25 or 26, characterized in that it the Euglena cells are cultivated as fed-batch cultivation.

28. The method according to claims 25 to 27, characterized in that an essentially bio-degradable surfactant selected from among non-ionic or anionic surfactants is used to isolate the paramylon from the Euglena cells.

29. The agent according to claims 1 to 3, characterized in that it serves for peroral administration.

30. The agent according to claim 29, characterized in that it comprises at least one compressed carrier, whereby the carrier has a sponge-like structure once it has expanded in the stomach.

31. The agent according to claims 1 to 3 or 30, characterized in that the carrier with the collagen structure comprises the amino acids glycine and hydroxyproline.

32. The agent according to claims 2 to 3 or 30 to 31, characterized in that the carrier stems from the phylum Porifera, especially the class of Demospongiae.

33. The agent according to claims 2 to 3 or 30 to 32, characterized in that, prior to its compression, the sponge-like carrier has a density ranging from 0.005 to 1 g/cm³, preferably from 00.1 to 0.1 g/cm³.

34. The agent according to claims 1 to 3 or 29 to 33, characterized in that the carrier is not encapsulated.

35. The agent according to claims 1 to 3 or 29 to 34, characterized in that the carrier has the form of a tablet, preferably an oblong tablet.

36. The agent according to claim 35, characterized in that the tablet has a soluble coating.

37. The agent according to claims 1 to 3 or 29 to 36, characterized in that the carrier also comprises at least one active ingredient and/or additive.

38. The agent according to claim 38, characterized in that the active ingredient is contained in a matrix, shell, embedding and/or in another carrier material that controls the release.

39. The agent according to claims 1 to 3, 29 to 33 and/or 37 to 38, characterized in that the carrier is encapsulated.

40. A method for the production of the agent according to claims 1 to 3 and/or 29 to 39, characterized in that a fine-pore, freeze-dried sponge having a density ranging from 0.005 to 1 g/cm³, which has optionally been treated with at least one active ingredient and/or additive prior to the compression procedure, optionally in the presence of a mold-release agent, is compressed to one-half to one-fiftieth, preferably one-third to one-thirtieth, of its original size and is optionally surrounded by a capsule that is soluble in gastric juice.

41. The method according to claim 40, characterized in that the compression procedure is carried out in at least one stage, preferably in at least two stages.

42. The method according to claim 40 or 41, characterized in that the fine-pore sponge is provided with a carrier layer for at least one active ingredient, in that the carrier layer is compressed in a known manner onto the pre-compressed sponge after the layer tablets have been produced.

43. The agent according to claims 1 to 3, characterized in that it serves for parenteral administration, especially as a depot implant.

44. Use of the agent according to claims 1 to 3 or of the agent that can be obtained according to claims 40 to 42 as a nutritional supplement, especially of vitamins, minerals, fatty acids and/or dietary fiber.

45. Use of the agent according to claims 1 to 3 or of the agent that can be obtained according to claims 40 to 42, for purposes of administering at least one, at least partially soluble, pharmacologically active ingredient, preferably with local or systemic effect, especially for modified active ingredient release.

46. Use of the agent according to claims 1 to 3 or of the agent that can be obtained according to claims 40 to 42, for the therapeutic or prophylactic treatment of diseases of the digestive tract, especially stomach diseases such as endogastritis.

47. Use of the agent according to claims 1 to 3 or of the agent that can be obtained according to claims 40 to 42, in a form that has a time of residence in the stomach.

48. An agent containing 1% to 80% by weight, preferably 5% to 75% by weight, of paramylon and 20% to 99% by weight, preferably 25% to 95% by weight, of the other carrier, namely, collagen, in the form of a freeze-dried, three-dimensional biomatrix, for purposes of the topical and/or parenteral administration and/or application of paramylon via body openings.
